(19) United States
(12) Patent Application Publication
(10) Pub. No.: US 2023/0165710 A1
(43) Pub. Date: Jun. 1, 2023

(54) OPHTHALMIC DRUG DELIVERY DEVICE

(71) Applicant: ContactRx LLC, Pasadena, CA (US)

(72) Inventors: Nicholas E. SCIANMARELLO, Monrovia, CA (US); Mark S. HUMAYUN, Glendale, CA (US); Yu-Chong TAI, Pasadena, CA (US); Andrew URAZAKI, Las Vegas, NV (US)

(21) Appl. No.: 18/072,385

(22) Filed: Nov. 30, 2022

Related U.S. Application Data
(60) Provisional application No. 63/284,376, filed on Nov. 30, 2021.

(51) Int. Cl. A61F 9/00

(52) U.S. Cl. CPC A61F 9/0017; A61F 2250/0068

(57) ABSTRACT

An ophthalmic liquid delivery apparatus includes a bulbar conjunctiva contacting surface, a palpebral conjunctiva contacting surface which is opposite the bulbar conjunctiva contacting surface, a therapeutic storage reservoir, and at least one fluid delivery port. In addition, a fill system for a liquid filled device includes a liquid feed tube, a plug rod feed tube, a junction port for the liquid feed tube and plug rod feed tube to meet, a needle output to temporarily interface with an ophthalmic liquid delivery device during the filling and plug step, and a cutting tool to cut the plug rod once the device is filled.

### CROSS-REFERENCES TO RELATED APPLICATION

This application claims the benefit of U.S. Provisional Application No. 63/284,376, filed Nov. 30, 2021, the contents of which are hereby incorporated in its entirety for all purposes.

### TECHNICAL FIELD

In various embodiments, the present invention relates generally to an ophthalmic device for administration of a therapeutic agent to the corneal or scleral surface of the eye. The invention incorporates various technologies to provide a non-electrical, non-propellant, long term duration drug delivery solution.

### BACKGROUND

The most common treatment modality for various eye conditions is through topical application of ophthalmic solutions administered as eye drops. This method of fluid (liquid, gas, gel, or combination of the aforementioned) delivery to the corneal and scleral surfaces of the eye accounts for a large percentage of all ophthalmic medications due to the simplicity, comparatively low cost, and ability to self-administer. However, this mode of delivery is very inefficient in many aspects. First, there is user error that causes a majority of eye drop volume to miss the corneal surface of the eye and wasted volume drops onto the eyelid or other facial region. Second, each eye drop volume is of a greater volume than the therapeutically required dose due to the calculated clearance rates of the drug affected by various factors including pre-corneal fluid drainage, drug binding to tear proteins, systemic drug absorption, and drug metabolism once exposed to ocular surface proteins, all of which have an effect on the intraocular bioavailability of the drug. Bioavailability of the drug administered by eyedrop usually ranges from two hours to twelve hours, thereby limiting the effective duration of treatment. Therefore, eye drops have a low compliance rate, minimal ability to adapt to long term use without vigilant re-administration, and high volumes of drug wasted. There are various ophthalmic drugs that can be used with the invention, however the greatest benefit would be liquid drugs to treat dry eye, allergy, and glaucoma along with any other ocular conditions that currently require repeated eye drop administration for disease management and lyophilized drugs that can be transitioned to a liquid state.

Various drug eluting options have been proposed including drug eluting contact lenses with drug embedded within a surface coating (hydrogel, polyethylene glycol (PEG)-based polymers), a drug eluting silicone tube placed in the ocular fornix, and concentrated degradable drug rod placed in the punctal plug or vitreous of the eye. However, these options usually require a highly concentrated version of the drug and correct interaction with the material into which the drug is embedded to ensure efficacious long term drug delivery through a diffusion process. These limitations restrict the duration of drug efficacy and the bioavailability of the drug due to varying concentrations of drug of first order kinetics in which the concentration of the drug is a determining factor. Furthermore, the process of creating a highly concentrated drug is very expensive and not always successful.

Ophthalmic drug delivery options are further limited by large designs required to maintain adherence to the cornea and/or sclera of the eye, therefore requiring a device that extends a large part of the circumference of the cornea and/or sclera such as a scleral contact lens or similar large surface area to be held in place by fluid stiction with the tear film fluid between the device and the eye. Such size makes the device noticeable regardless of efforts to improve comfort through the use of flexible materials, soft coatings, and hydration improving materials. Even with such large size, contact lenses, scleral lenses, and other devices with main interaction with the eye are prone to shifting during movement.

Existing medical equipment actuation mechanisms and metering systems that rely on electrical or propellant actuation would be very difficult if not impossible to miniaturize into a scale that is comfortable to place as a contact lens or onto the scleral surface while providing a large enough drug reservoir volume for long term duration efficacy.

Considering the above mentioned various limitations of existing approaches, there is a need in the field for an improved approach of ophthalmic drug delivery.

### SUMMARY

In various embodiments, the present invention relates generally to an ophthalmic drug delivery device designed to be placed primarily in the superior or inferior conjunctival fornix of the human eye. The conjunctival fornix is comprises of the palpebral conjunctiva anteriorly and the bulbar conjunctiva posteriorly. The device ideally has minimal surface interaction with the corneal and/or scleral surface of the eye once placed within the conjunctival fornix with a majority of the device nested within the conjunctival fornix. By having minimal interaction with the corneal and scleral surfaces, the ocular itch and redness caused by hyperemia are avoided which are common signs of ocular irritation. Therapeutic released by the device travels to surrounding tissues including but not limited to: the conjunctiva, cornea, sclera, anterior chamber, and posterior chamber.

### Controlled Fluid Delivery

The accurately controlled drug delivery flow rates of these devices can be achieved by using various methods. The design of a flexible, yet durable fluidic resistor provides sufficient restriction such that the average flow throughout the useable active time of the device has a rate between 0.01 μL/hr and 5 μL/hr, and more ideally between 0.05 μL/hr and 2 μL/hr depending on dosing requirements of a specific drug to provide therapeutic efficacy. The fill volumes range from 1 μL to 200 μL, and more ideally from 54, to 754, to allow the device to have a continuous useable range from 1 day to 1 month (e.g. 0.16 μL/day with a 5 μL reservoir or 2.5 μL/day with a 75 μL, reservoir provides a 30 day reservoir) while being easily and comfortably worn by the patient. The optimal duration would be based upon the target indication, drug volume required for efficacy based on pharmacokinetic and pharmacodynamic characteristics of the drug, drug stability in the range of 25° C. and 40° C., and optimal continuous drug exposure limitations. For example, for an allergy drug, the device would continue to deliver a therapeutic dose optimally for one week or two weeks to coincide with the changing of a weekly or bi-weekly use contact lens.

There are multiple embodiments for the fluidic resistor in this application. In a first embodiment, a narrow microchannel with either a round cross section, square cross section, or other shape that holds its cross-sectional shape with minimized tendency of the cross sections collapsing when the material is flexed during handling or while integrated within the device. The narrow microchannel can be produced either by placing a microwire of hard metal or thermally decomposable plastic (e.g. diameters of 5 μm, 10 μm, 20 μm, etc.) within the casting mold for the lens shape or by adding a modular resistor manufactured through VLSI based machining of thermally decomposable plastic, hot embossing of a micro pattern or by stamping and casting. The microwire approach results in rounded cross section and a simplified manufacturing process. The wire can either be removed post casting or by thermally decomposing if made of a material such as poly(ethylene carbonate)(C3H4O3)(e.g. QPac®25 or QPac®40 (manufactured by Empower Materials Inc. Delaware, USA). These methods result in smaller channel lengths with circular channel diameters between 5 μm and 50 μm. These generally square, circular, or half-round cross sectioned channels can be manufactured to have a width between 5 μm and 200 μm, and a height between 5 μm and 50 μm. The channels can bend tightly adding centimeters of total length within a small area in accurately reproducible cross sections and diameters. Such channels can be created by etching thermally decomposable plastic, such as QPac®25, and then capping before decomposing the plastic in vacuum. The channels can also be produced via hot embossing, stamping, etching, or molding over a lithographically patterned channel using various microelectromechanical systems (MEMS) processes.

In various embodiments, the fluidic resistor is in the shape of a flexible disk to allow for modular placement within the device. In certain embodiments, two or more flexible fluidic resistor disks can be stacked if the fluidic inlet and outlet of adjacent fluidic resistor disks are configured to link in series, thereby creating a longer total fluidic resistor.

In another embodiment, a microchannel can be packed with microbeads. To further increase the resistance per length of these microchannels the channels can be packed with beads which help obstruct flow. The beads can be added as a dry media or in a liquid state in channels formed as described above by ensuring the channel has a throat portion that restricts the size to below the radius of the smallest packed beads. Columns can also be manufactured within the channel with column-to-column distances being less than the size of the microbeads. Similarly, media of different sizes can be used where larger beads are packed towards the throat and smaller media behind those beads allowing for a larger throat to still use smaller media. The media can also be added to the thermally decomposable plastic either by mixing with the liquid solution applied before lithography and etching or by spraying or direct application after the liquid solution has solidified and before lithography. Similarly, in hot embossing, stamping, or molding a pellet of beads suspended in thermally decomposable plastic can be added to the channel during manufacture. After thermal cycling the beads are freed and the first fluid flow through the channel serves to pack them.

In yet another embodiment, a hydrophilic or hydrophobic porous membrane can be used as a fluidic resistor. In such a case, the fluidic resistance is best described as diffusion in which case the relative concentration on either side of the membrane dominates the flow. Since liquid phase drugs are incompressible their concentration for liquid mass will be fixed with respect to pressure. The reservoir becomes a equal concentration source and rapid diffusion through the membrane is determined by the relative concentration. In such a case high flow restrictions can be achieved. This embodiment is beneficial for viscous drugs, or drugs that have a tendency to generate aggregates that could potentially clog microchannels or adhere to beads that would reduce the flow rate over time. By having a larger surface area porous membrane, clogging of a small relative surface area of the porous membrane has minimal effect on the flow rate.

Any of the above fluidic resistors types can be made with great flexibility in channel length and therefore flowrate by manufacturing such resistors in a modular fashion. By adding one or more microfluidic resistor chips that are manufactured separately in series or in parallel, the flow rate can be accurately controlled. This microfluidic chip can be coupled to the reservoir which can be defined using a thermally decomposable plastic by cementing them with the same dissolved plastic. This ensures a connected fluidic path after thermal decomposition.

One of the main considerations of the fluidic resistor is the ability to place the resistor at or within the fluid delivery port, thereby maximizing the drug reservoir volume. In another embodiment, where the device has two or more fluid delivery ports, the device has a resistor embedded at each fluid delivery port, thereby releasing fluid to two separate tissue areas simultaneously. Such embodiment is beneficial in many embodiments including delivery of ocular lubrication to the eye or drug delivery toward the corneal surface from two or more radial outputs to increase permeation. Each of the fluid delivery ports are temporarily sealed during transport by a removal plug, film, or foil. Such removable mechanism is beneficially attached to the packaging and simultaneously opens fluid delivery ports upon opening of the package. Such packaging may be similar to a blister pack as commonly used for individually packaging daily contact lenses in a liquid solution, thereby keeping the device hydrated during transport as to not modify the material properties of the device.

Although the one or more fluid delivery ports can be placed either on the medial or lateral side of the eye, due to the tear ducts (e.g. nasolacrimal duct and connected canaliculi and puncta) being located medially towards the nose, the fluid delivery port is ideally on the lateral side to ensure the drug released from the device does not get quickly drained away.

### Fill Port and Method of Filling Device

The device additionally consists of a fill port which is used to initially fill the device's drug reservoir with fluid. The drug reservoir is made of a flexible material such as medical grade silicone, thereby allowing the device to be filled until taut and even pressurized to 1 psi-3 psi without rupturing. Filling until a specific fluid pressure is achieved ensures initial flow out of the one or more fluid delivery ports and continuous flow thereafter. Once the device is worn, continuous drug flow is achieved by a combination of the initial pressurization of the drug reservoir, constant pressure from adjacent tissues (e.g. relative positioning of device in between the eye and eyelids and the action of blinking through which the eyelids press against the flexible drug reservoir). Placement of the device within the fornix between the palpebral conjunctiva and bulbar conjunctiva further helps in applying a constant pressure to the drug reservoir.

The fill port can be in various embodiments to suit the embodiment specific needs of drug viscosity, flow rate, pressurization, and fill requirements. In one embodiment, the fill port is an elastomeric fill port that is accessible by a conventional needle. Upon removing the needle, the elastomeric properties tied with the radial and axial forces of the fill port closes the temporary aperture made by the needle. In another embodiment, a specialized needle syringe is used that contains a removable plug rod within. The needle with removable plug rod is inserted into the fill port of the device. The fluid plunger of the needle syringe is depressed in one stage to force fluid from the needle syringe through the device's fill port and into the device's drug reservoir. In a secondary stage of plunger depression, the removable plug rod is pushed out of the needle and embeds into the fill port of the device, thereby sealing the fill port. The fill port may benefit from having a sealing channel within with a radius slightly less than that of the plug rod. The sealing channel may further have a conical taper to allow the plug rod to be securely inserted through the fill port. As a flexible device, the plug rod itself is also ideally a flexible material such as a cured higher durometer silicone.

For mass production purposes, the devices may be filled by an aseptic fill process that incorporates both drug fill and plug rod insertion system. In one embodiment, the plug rod insertion system is completed by a wire feed system. Devices can be systematically filled by cycling through the steps of: 1) drug pumping into the device through the fill port to fill the device's drug reservoir, 2) advance plug rod through a wire feed system, 3) cut device fill port tab and plug. Such automation system will allow for devices to be mass produced.

In certain embodiments, the device consists of one or more secondary drug reservoirs. Each drug reservoir may be fluidically connected to a single fluid delivery port or paired to a separate fluid delivery port. Each drug reservoir may have a separate fluidic resistor so as to allow each drug reservoir and drug combination to have a specific flow rate sustainable for the anticipated treatment duration of the device and subsequently a separate fluid delivery port.

In many embodiments, the device has a corneal edge that is positioned near the cornea and a scleral edge that is positioned away from the cornea towards the sclera. The scleral edge extends into the eyelid toward the conjunctival fornix. Each edge is tapered to be comfortable, yet durable to limit tearing from such edges while handling. In embodiments where the device fits under the eye lid, the anterior surface interfaces with the palpebral conjunctiva while the posterior surface interfaces with the sclera and bulbar conjunctiva. Due to the shape, size, and gravitational orientation when the user is in the standing position, the device ideally sits in the lower eye lid near the inferior fornix. The device is easily placed in the lower eye lid by pulling down on the lower eyelid and dropping the device in. The device is easily removed by pulling down on the lower eyelid, thereby visually exposing the device. As one of the ends of the device is exposed, the device easily releases itself from within the inferior fornix and can be removed.

In certain other embodiments, the device may be configured to be placed under the upper eye lid. However, due to the movement of the upper eyelid being greater during blinking and gravitational pull on the device downward, the device is ideally placed under the lower eyelid.

The device can have different radial shapes ranging from 40 degrees to 360 degrees, and more ideally radial shapes ranging between 60 degrees and 330 degrees to closely adhere to the scleral surface of the eye which varies in the general human population from 21 mm to 28 mm in diameter. In embodiments for which the device sits in the lower eye lid near the inferior fornix when worn, the radial shape is ideally between 60 degrees and 170 degrees.

In yet another embodiment, one or more bumps, lines, or other friction increasing sections are added to the device surface interfacing with the eyelid. By increasing the friction on the eyelid side only, the device remains in relatively the same position while the eye moves during gaze direction change, and even during sleep during which the Bell's phenomenon (palpebral-oculogyric reflex) moves the eyes in an upward direction.

All of the above mentioned components including the drug reservoir, modular flow resistor, and fill port are manufactured with a curvature that matches the scleral surface of the eye, thereby making a low profile (less than 1.5 mm) device possible. Each component is made of flexible materials, allowing for adaptation to the curvature of the wearer's eye and positive retainment thereto. In many embodiments a medical grade silicone is used due to a combination of biocompatibility, durability, and cost. However, other flexible and durable materials may be substituted.

In another embodiment, the corneal edge and scleral edge of the device are tapered to 0.2 mm. The distance between the corneal edge and scleral edge is 5 mm, thereby extending into the conjunctival fornix to prevent dislodging when blinking or during ocular movement. The thickest portion of the device, the drug reservoir is 1 mm, thereby fitting between the palpebral conjunctiva and bulbar conjunctiva. The fluid delivery port and fill port regions near the ends of the device are 0.2 mm in thickness.

To improve comfort of the device, various methods and materials used in commercial contact lenses may be used to improve wetting of the external surface. Common processing for soft contact lenses includes plasma treatment of the surface to improve adhesion of a silicone hydrogel layer which is overmolded to surround the device. The hydrogel layer may alternatively be any water-gradient structural configuration that creates a soft, water-rich, and lubricious surface that greatly improves comfort during long term wear. The hydrogel layer formulation varies by manufacturer but commonly used formulations include: Nelficon A, Lotrafilcon A, and Verofilcon A to name a few.

### Adaptation of Device to Other Forms of Therapeutics

In embodiments where the therapeutic is in the form of a lyophilized drug or drug pellet, the device can be adapted to rehydrate the drug within the therapeutic storage reservoir. The walls of the storage reservoir are made of materials such as silicone that allow water vapor (i.e. component of tear film from the ocular environment) to pass into the reservoir through osmosis and rehydrate the lyophilized drug within the reservoir. Downstream of the therapeutic storage reservoir are hydrogel pathways that prevent the lyophilized drug from traveling downstream without first being rehydrated. One hydrated, the drug moves through the downstream hydrogel pathways toward the fluid delivery port. The flow rate is determined by the length and cross section of the photo-defined hydrogel paths and the concentration of the hydrated drug. The concentration is defined by the balance between the osmotic pressure and the pressure generated by the hydrated drug expanding within the reservoir. Optionally, additional flow rate restriction can be placed within the flow path by including the modular flexible flow restrictor as described above.

One exemplary embodiment of manufacturing the lyophilized drug within the therapeutic storage reservoir is provided below. In the first step, a single layer of silicone is spun and cured in an appropriate shape. In the second step, a hydrogel with photo-initiator is placed as a second layer on top of the silicone. In the third step, a photomask of a specific pattern is placed on the hydrogel to define the specific shape of the hydrogel to be crosslinked. The non-masked hydrogel area is crosslinked using ultraviolet or other known methodologies of crosslinking the hydrogel. The photomask is removed. The drug pellet of lyophilized drug is placed in the desired crosslinked hydrogel area. Finally, a secondary layer of silicone is deposited to create an encapsulated reservoir containing the drug pellet.

In yet another embodiment, the therapeutic containing reservoir and downstream flow path may be coated with a hydrophobic, hydrophilic, lipophilic, or lipophobic layer to better suit various fluid based drugs including oil emulsion based drugs.

These and other objects, along with advantages and features of the present invention herein disclosed, will become more apparent through reference to the following description, the accompanying drawings, and the claims. Furthermore, it is to be understood that the features of the various embodiments described herein are not mutually exclusive and may exist in various combinations and permutations. As used herein, the terms “approximately” and “substantially” mean±10%, and in some embodiments, ±5%. The term “consists essentially of” means excluding other materials that contribute to function, unless otherwise defined herein. Nonetheless, such other materials may be present, collectively or individually, in trace amounts. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIGS schematically illustrate components of a representative embodiment.

FIG. 1 illustrates a front view of an ophthalmic liquid delivery apparatus.

FIG. 2 illustrates a bottom view of an ophthalmic liquid delivery apparatus.

FIG. 3A illustrates a right side view of an ophthalmic liquid delivery apparatus in a non-filled state.

FIG. 3B illustrates a right side view of an ophthalmic liquid delivery apparatus in a filled state.

FIG. 4 illustrates a left side view of an ophthalmic liquid delivery apparatus.

FIG. 5 illustrates a back view of an ophthalmic liquid delivery apparatus.

FIG. 6 illustrates an isometric view of an ophthalmic liquid delivery apparatus.

FIG. 7 illustrates a front view of an ophthalmic liquid delivery apparatus with integrated foil cover.

FIG. 8A illustrates a top view modular flexible flow restrictor.

FIG. 8B illustrates a cross sectional view of a modular flexible flow restrictor.

FIG. 9A-D illustrates a fill port being accessed by a custom fill needle and the subsequent fill procedure, plugging of the fill port, and removal of the needle.

FIG. 10 illustrates an embodiment of an automated fill system.

FIG. 11 illustrates the process map of an embodiment of an automated fill system and indexed device holder.

FIG. 12A-E illustrates the cross section of the process of creating a lyophilized drug pellet and cross-linked hydrogel within the therapeutic storage reservoir.

FIG. 13A-E illustrates the top view of the process of creating a lyophilized drug pellet and cross-linked hydrogel within the therapeutic storage reservoir.

### DETAILED DESCRIPTION

Generally, ophthalmic liquid delivery apparatuses are described herein. In various embodiments, the present invention relates generally to an ophthalmic drug delivery device designed to be placed primarily in the superior or inferior conjunctival fornix of the human eye.

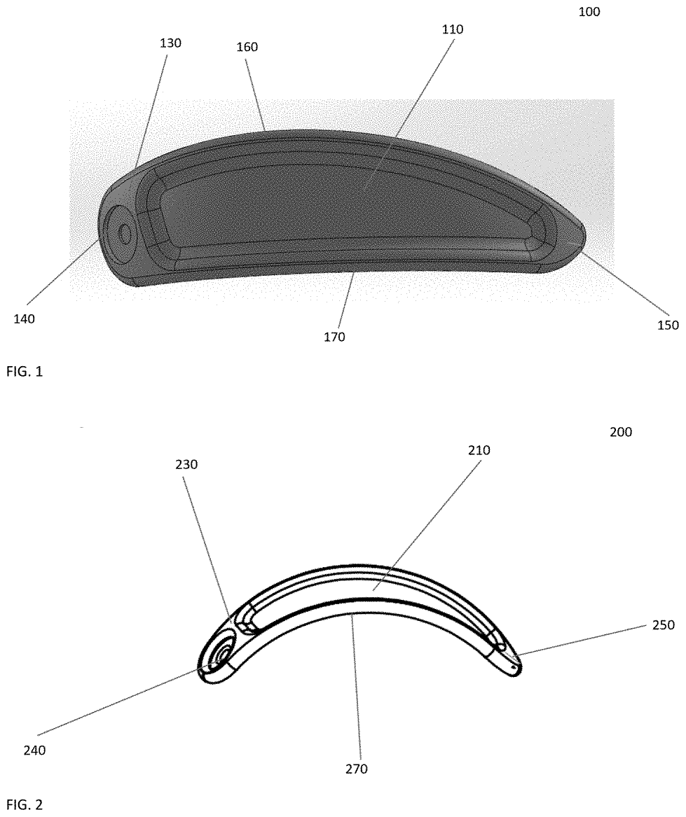

FIG. 1 illustrates a front view of an ophthalmic liquid delivery apparatus. Ophthalmic liquid delivery apparatus 100 includes a therapeutic storage reservoir 110, a fluid delivery port 140, and a fill port 150 within the device. The device is further defined by a palpebral conjunctiva contacting surface, a bulbar conjunctiva contacting surface (not shown), an upper edge 160 of the device, and a lower edge 170 of the device. The perimeter including the edges and portions adjacent the fluid delivery port 140 and fill port 150 are tapered to improve comfort of the device and durable enough to withstand the sheer stresses.

FIG. 2 illustrates a bottom view of an ophthalmic liquid delivery apparatus.

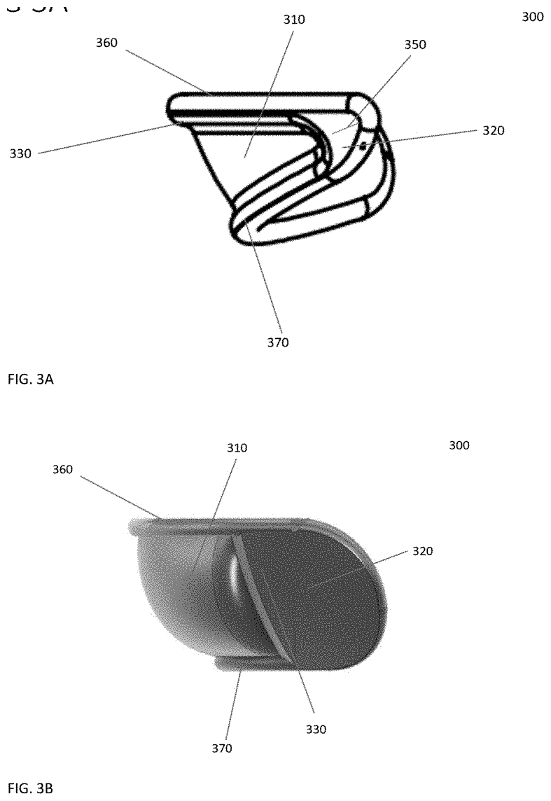

FIG. 3A illustrates a right side view of an ophthalmic liquid delivery apparatus in a non-filled state. The walls of the therapeutic storage reservoir 310 is flexible and slowly collapses as the drug is expelled. Therefore, the approximate fill amount can be confirmed visually and by palpitation of the central portion of the device.

FIG. 3B illustrates a right side view of an ophthalmic liquid delivery apparatus in a filled state. The therapeutic storage reservoir 310 is flexible and is in the expanded state. Due to the shape and size of the fornix, the thickness of the therapeutic storage reservoir 310 walls can be adjusted to predominately favor expansion toward the palpebral conjunctiva contacting surface 330.

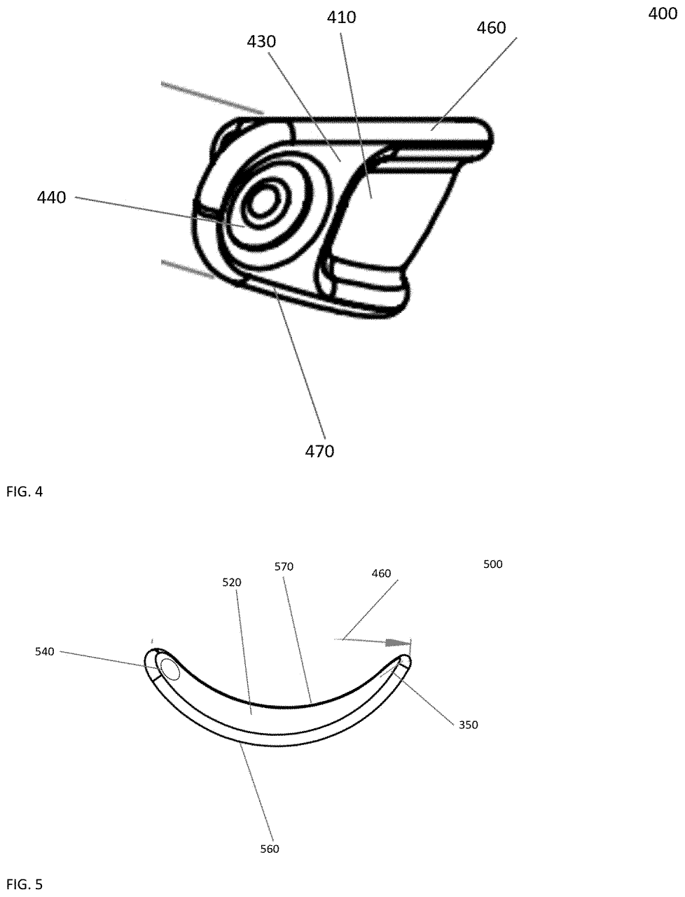

FIG. 4 illustrates a left side view of an ophthalmic liquid delivery apparatus.

FIG. 5 illustrates a back view of an ophthalmic liquid delivery apparatus.

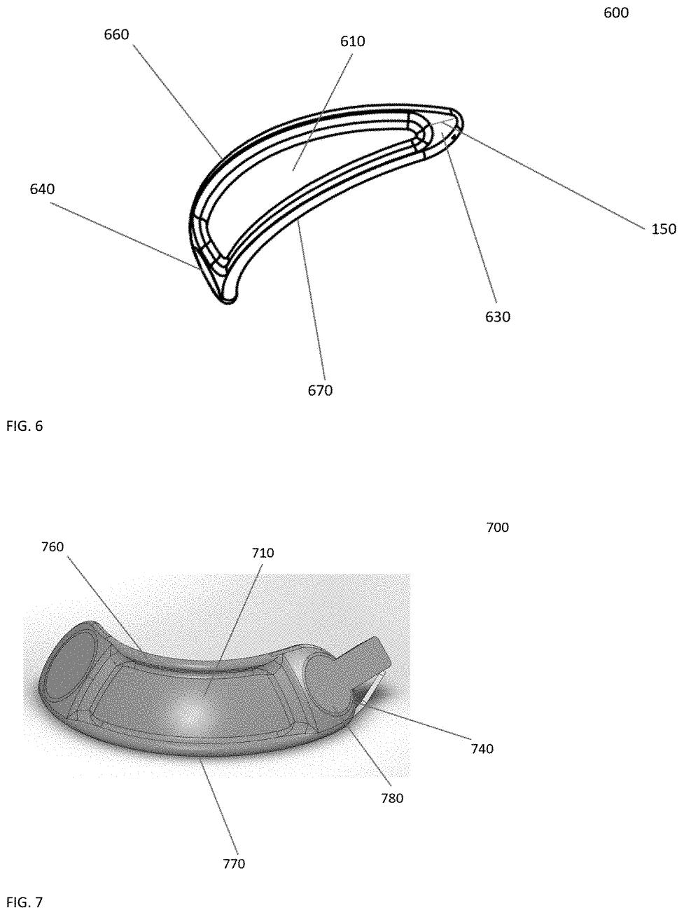

FIG. 6 illustrates an isometric view of an ophthalmic liquid delivery apparatus.

FIG. 7 illustrates a front view of an ophthalmic liquid delivery apparatus with integrated foil cover.

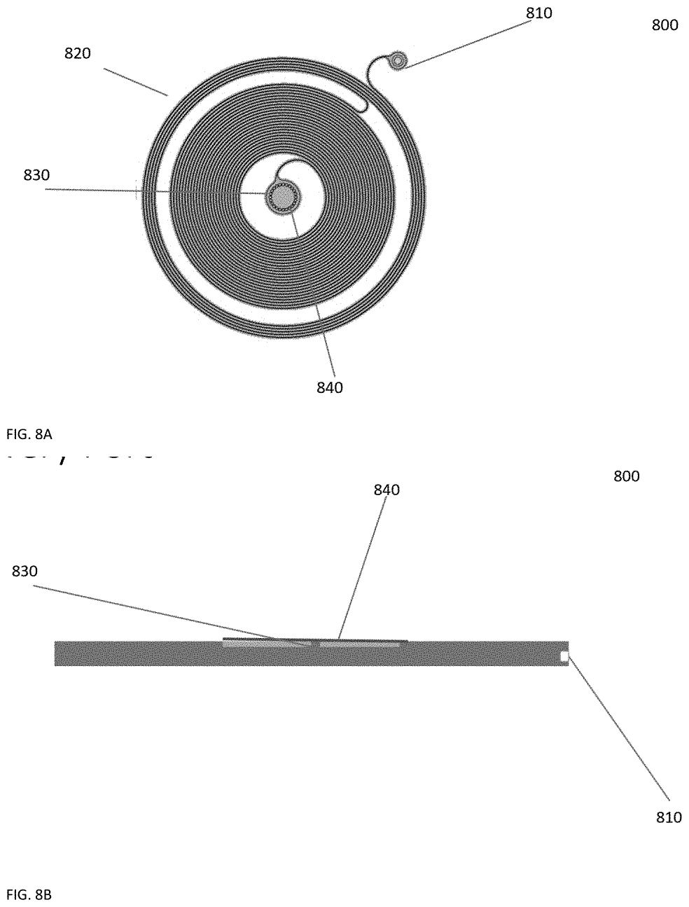

FIG. 8A illustrates a top view modular flexible flow restrictor 700. The modular flexible flow restrictor 700 comprises the following components: fluidic connector 710 to therapeutic storage reservoir, fluidic channel 720 for flow restriction, fluid delivery port 730. Optionally, an anti-bacterial membrane filter 740 is placed on the fluid delivery port 730 to ensure there is no bacterial ingrowth from adjacent ocular tissues. The fluid delivery port 730 preferably has multiple exit ports or is in the shape of a large surface area exit port to prevent protein clogging during the duration of device use.

FIG. 8B illustrates a cross sectional view of a modular flexible flow restrictor in which the placement of the anti-bacterial membrane filter 740 can be seen.

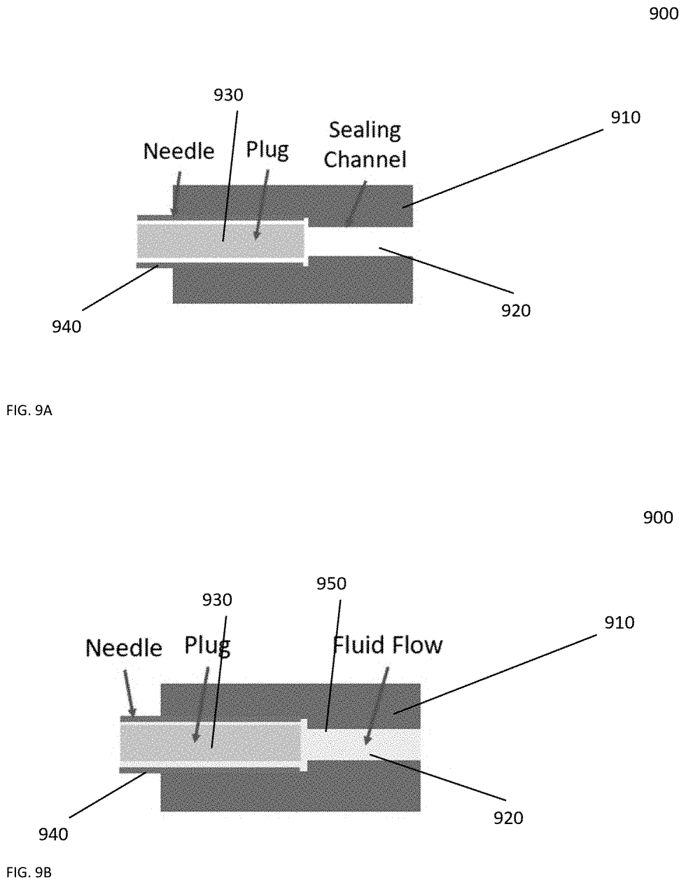

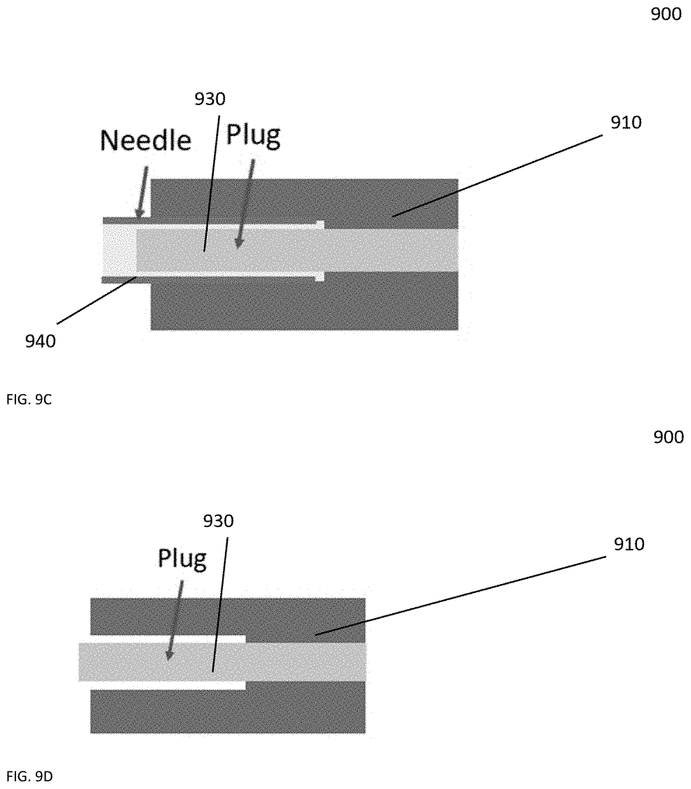

FIG. 9A-D illustrates a fill port being accessed by a custom fill needle and the subsequent fill procedure, plugging of the fill port, and removal of the needle.

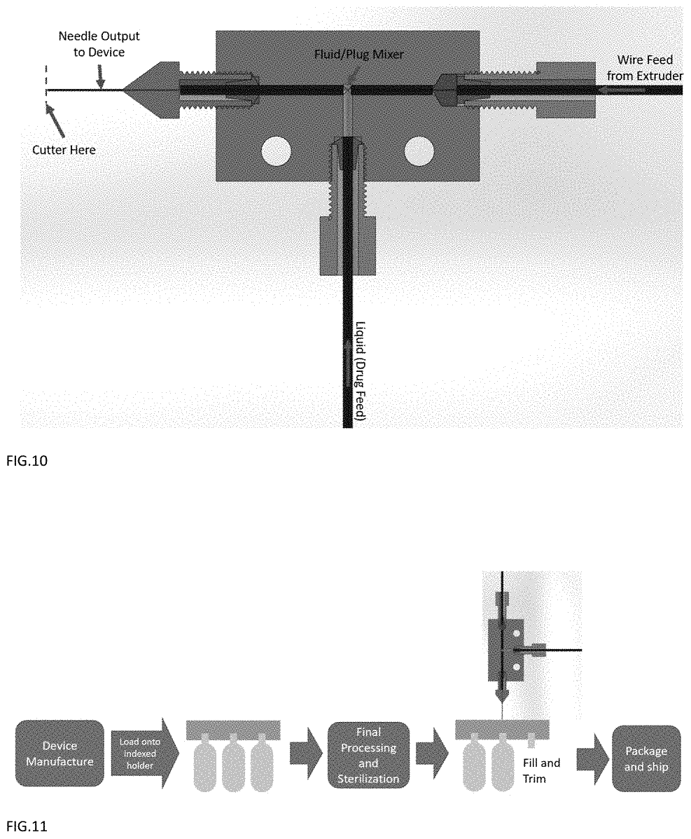

FIG. 10 illustrates an embodiment of an automated fill system.

FIG. 11 illustrates the process map of an embodiment of an automated fill system and indexed device holder.

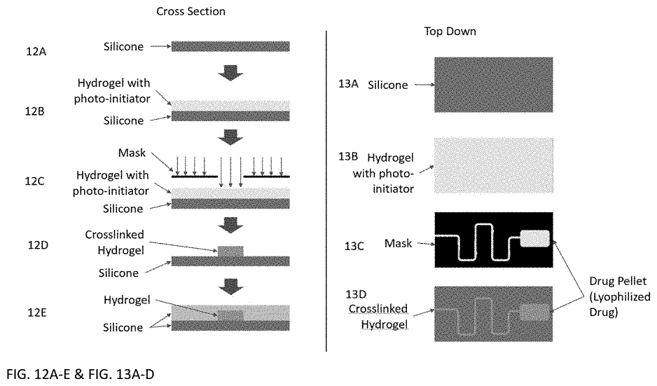

FIG. 12A-E illustrates the cross section of the process of creating a lyophilized drug pellet and cross-linked hydrogel within the therapeutic storage reservoir. One exemplary embodiment of manufacturing the lyophilized drug within the therapeutic storage reservoir is provided below. In the first step, a single layer of silicone is spun and cured in an appropriate shape. In the second step, a hydrogel with photo-initiator is placed as a second layer on top of the silicone. In the third step, a photomask of a specific pattern is placed on the hydrogel to define the specific shape of the hydrogel to be crosslinked. The non-masked hydrogel area is crosslinked using ultraviolet or other known methodologies of crosslinking the hydrogel. The photomask is removed. The drug pellet of lyophilized drug is placed in the desired crosslinked hydrogel area. Finally, a secondary layer of silicone is deposited to create an encapsulated reservoir containing the drug pellet.

FIG. 13A-E illustrates the top view of the process of creating a lyophilized drug pellet and cross-linked hydrogel within the therapeutic storage reservoir.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive.

1. An ophthalmic liquid delivery apparatus comprising:
a bulbar conjunctiva contacting surface;
a palpebral conjunctiva contacting surface which is opposite the bulbar conjunctiva contacting surface;
a therapeutic storage reservoir; and
at least one fluid delivery port.

2. The apparatus of claim 1, wherein the ocular surface contacting surface has the same curvature as the sclera of the human eye.

3. The apparatus of claim 1, wherein the at least one fluid delivery port comprises a modular flow resistor to control the flow rate.

4. The apparatus of claim 1, further comprising a fill port.

5. The apparatus of claim 1, further comprising a therapeutic liquid within the therapeutic storage reservoir.

6. The apparatus of claim 1, further comprising a lyophilized therapeutic within the therapeutic storage reservoir.

7. The apparatus of claim 6, wherein the bulbar conjunctiva contacting surface allows for water vapor to pass into the therapeutic storage reservoir.

8. The apparatus of claim 6, wherein the palpebral conjunctiva contacting surface allow for water vapor to pass into the therapeutic storage reservoir.

9. The apparatus of claim 3, wherein the modular flow resistor's fluid pathway consists of a flexible material.

10. The apparatus of claim 1, wherein the thickest portion is the therapeutic liquid storage reservoir with a thickness less than 1.5 mm.

11. The apparatus of claim 1, wherein the therapeutic liquid storage reservoir has a curvature to secure the device within the superior or inferior conjunctival fornix of the human eye.

12. The apparatus of claim 1, wherein the surface opposite the ocular surface contacting surface comprises one or more protrusions to help secure the device against the palpebral conjunctiva.

13. The apparatus of claim 1, further comprising an over-molded layer of silicone hydrogel.

14. The apparatus of claim 13, wherein the over-molded layer of silicone hydrogel has one or more channels to allow for water vapor diffusion.

15. The apparatus of claim 1, wherein the fluid delivery port further comprises an anti-bacterial membrane filter.

16. The apparatus of claim 1, wherein the apparatus is asymmetrical when comparing the medial nasal portion vs. the lateral portion of the apparatus.

17. The apparatus of claim 1, further comprising a one-way check valve between the therapeutic storage reservoir and the fluid delivery port.

18. The apparatus of claim 1, further comprising a variable resistance check valve between the therapeutic storage reservoir and the fluid delivery port.

19. A fill system for a liquid filled device comprising:
a liquid feed tube;
a plug rod feed tube;
a junction port for the liquid feed tube and plug rod feed tube to meet;
a needle output to temporarily interface with an ophthalmic liquid delivery device during the filling and plug step; and a cutting tool to cut the plug rod once the device is filled.

20. The fill system of claim 19, further comprising an indexed holder to serially bring subsequent ophthalmic liquid delivery devices in temporary contact with the needle of the fill system during fill and seal process of the ophthalmic liquid delivery devices.
